(19) United States
(12) Patent Application Publication
(10) Pub. No.: US 2022/0312800 A1
(43) Pub. Date: Oct. 6, 2022

(54) APPARATUS FOR AND METHOD OF STEAM TREATING CANNABIS

(71) Applicant: HAYGAIN LTD, Lambourn, Berkshire (GB)

(72) Inventors: Becky WINDELL, Lambourn, Berkshire (GB); James MILES, Lambourn, Berkshire (GB); Edzo WISMAN, Lambourn, Berkshire (GB)

(73) Assignee: HAYGAIN LTD, Lambourn, Berkshire (GB)

(21) Appl. No.: 17/640,525

(22) PCT Filed: Sep. 4, 2020
(86) PCT No.: PCT/GB2020/052133

§ 371 Date: Mar. 4, 2022

(30) Foreign Application Priority Data
Sep. 6, 2019 (GB) 1912868.5

(51) Int. Cl. A23L 3/00; A61L 2/07

(52) U.S. Cl. CPC A23L 3/001; A23L 3/003; A61L 2/07

(57) ABSTRACT

An apparatus for steam treating cannabis is provided, the apparatus comprising: a steam manifold (1) that is adapted to receive steam from a steam source and to distribute steam to a plurality of lances, the lances being configured to deliver steam into the interior volume of a batch of cannabis (10); a temperature sensor for sensing a temperature of the batch of cannabis, and a controller. The controller is configured to receive a signal from the temperature sensor (27), and to control the supply of steam to the lances to raise the temperature of the cannabis to a target temperature. A method of steaming cannabis is also provided.

### FIELD

The invention relates to an apparatus for and method of steam treating cannabis, in particular cannabis for human consumption for medicinal or recreational purposes. The apparatus is particularly suitable for steam treating batches of harvested cannabis in compressed or uncompressed forms, for example cannabis in baled form, which is intended to include bales as well as retaining nets, bags, baskets or similar receptacles.

### BACKGROUND

The use of the Cannabis sativa plant and its various derivatives for medicinal purposes has become more widespread in recent years, and a variety of cannabis-derived substances are now legal for medicinal purposes in a large number of countries around the world. Recreational use of cannabis has also been legalised in a number of countries.

As the legal market for cannabis and its various derivatives has become more geographically widespread, there is a need for methods of farming and processing cannabis plants to be scalable for higher capacities and to meet high standards of hygiene.

When cannabis crops are harvested, the stems, flowers, buds and leaves of the cut plants may be separated and compressed into bales for ease of storage or transport. These bales of cut plants typically contain leaf shatter, soil, mesophilic moulds, thermophilic moulds, plant particles, fragments of sundry inorganic materials, bacteria, fungi and fungal spores, insects, and other organisms in varying amounts. All of this matter is generally classified as dust. Much of this dust is present in particles of less than 5 microns in diameter (respirable particles) and these particles can cause allergic reactions and respiratory problems in humans. When the plants have been cut and stored additional organisms (thermophilic actinomycetes) may also be present, which is a particular concern in the portions of the cannabis plant destined for human ingestion.

Aspergillus (thermophilic mould) is a very common mould that can be found on more than just the buds of the plant. When found in high concentrations in the air, Aspergillus spores can wreak havoc on those with damaged lungs or weakened immune systems. It is commonly found in soil or anywhere with a high moisture content, including the buds of a cannabis plant. Unlike bud rot, Aspergillus can affect cannabis plants during storage.

Cannabis contaminated with high levels of Aspergillus can result in harmful infections. High concentrations can lead to the development of an aspergilloma, which can cause symptoms ranging from a basic cough to severe fatigue and hemoptysis. To those with weakened immune systems, Aspergillus exposure can turn into invasive pulmonary aspergillosis, which can spread to other parts of the body besides the lungs, and is capable of resulting in death if not treated properly.

As a result of these health risks, exposure of a cannabis crop to mould typically renders the crop un-usable. Ingesting a cannabis product contaminated with mould can cause serious harm to the consumer, especially if they have a compromised immune system. Cannabis contaminated with mould should therefore be discarded in its entirety to protect consumer health. As cannabis becomes more widely used as a medicinal product, the control of mould and other contaminants is of utmost importance.

It would be desirable to clean harvested cannabis material to get rid of these harmful contaminants, without damaging or degrading the high-value crop.

### SUMMARY OF THE INVENTION

According to a first aspect of the invention there is provided apparatus for steam treating cannabis, the apparatus comprising:

a steam manifold that is adapted to receive steam from a steam source and to distribute steam to a plurality of lances, the lances being adapted for delivering steam into the interior volume of a batch of cannabis;

a temperature sensor for sensing a temperature of the batch of cannabis; and

a controller configured to receive a signal from the temperature sensor, and to control the supply of steam to the lances to raise the temperature of the cannabis to a target temperature.

The apparatus of the present invention may advantageously provide a solution for sterilising cannabis, in particular by killing thermophilic and mesophilic mould spores, bacteria and thermophilic actinomycetes that are prone to grow on cannabis plants.

A batch of cannabis is intended to refer herein to a batch of harvested cannabis material obtained from Cannabis sativa plants, for example a batch of cut cannabis plants, or a batch of cannabis flowers, cannabis buds, cannabis leaves, and/or cannabis stems. The harvested cannabis material may be in baled form, for example in bales or bricks, or in nets, mesh bags or containers.

The harvested cannabis material may be in compressed form, such as compressed bales or bricks.

The harvested cannabis material may be in loose, uncompressed form, for example as a net, bag or container of loose cannabis.

Preferably the cannabis is cannabis for human consumption for medicinal or recreational purposes.

The present invention may be particularly suitable for steam treating the parts of the Cannabis sativa plant that are intended for human consumption for medicinal or recreational purposes, either directly or after processing into cannabis-derived products. These parts of the plant, which typically contain the highest proportion of cannabinoid (for example the flowers or buds) tend to have a low moisture-tolerance and may be prone to degrade in damp conditions.

Steam treating has previously been employed as a technique to kill such spores and microorganisms in bales of animal fodder, and at the same time to dampen the fodder to reduce the proportion of airborne particles respirable by animals. A prior art steaming apparatus is disclosed in European patent EP2364100B1 of Haygain Limited, for example.

While it is desirable to process cannabis to kill any harmful pathogens, it is undesirable to place cannabis flowers or buds in an overly moist environment, as doing so may undesirably degrade the quality of the flowers or buds and therefore devalue the crop.

In order to avoid excessive dampening of the cannabis during steaming, the apparatus of the present invention may employ a temperature sensor and a controller configured to receive a signal from the temperature sensor. This advantageously allows improved control of the steaming process compared to the devices known in the prior art. Whereas in prior art devices the steaming process has been relatively simple and not actively controlled, the controller and temperature sensor of the present invention allow improved control of the temperature to which the cannabis is heated during steaming. This may be particularly advantageous when steaming temperature-sensitive cannabis, where excessive temperatures may cause undesirable degradation of the plant material.

The temperature sensor may preferably be provided on a probe insertable into the batch of cannabis during steaming.

In the prior art steamer disclosed in European patent EP2364100B1 of Haygain Limited, for example, steam is typically continuously delivered to a compacted bale of fodder to cause condensation that raises the moisture content of the fodder and thus greatly increases its heat capacity. The temperature of the baled fodder is increased exponentially during the continuous steam delivery, so that harmful pathogens are killed, and the resulting fodder is dampened prior to consumption by animals.

When steam treating cannabis, particularly the delicate flowers and buds of the cannabis plant, it is desirable to obtain the benefit of the sterilisation while dampening the plant to a much lesser degree than is desirable for animal fodder.

By monitoring the temperature of the cannabis during steaming, and controlling the steam supply to raise the temperature of the cannabis to a target temperature, the present apparatus may advantageously avoid supplying more steam than is necessary. This means that the steamer can heat the cannabis to the target temperature required to kill harmful organisms, without over-heating and over-moistening the cannabis with excess steam. This may advantageously reduce heat and moisture-related degradation of the cannabis plant material.

In order to steam clean cannabis to reduce mold/bacteria, the controller is preferably programmed to control the steam supply so that the temperature of the cannabis quickly reaches no less than 90° C. for a minimum of 10 minutes and no more than 104° C. for a maximum of 30 minutes.

The apparatus may comprise a valve or valve means controllable to prevent or allow the passage of steam from the steam source to the lances, and in which the valve or valve means are controllable by the controller.

The use of a valve or valve means between the steam source and the lances may advantageously allow the controller to stop and start the flow of steam to the lances during steaming.

The valve or valve means may allow the controller to generate steam pressure in the steam source prior to opening the valve or valve means, so that steam is delivered to the lances and into the plant material at a pressure greater than atmospheric pressure. This may advantageously mean that the steam is delivered at high pressure and the cannabis plant material is heated to a target temperature as quickly as possible. By heating the cannabis plant material more quickly, this may advantageously reduce the length of time during which the steam must be delivered to the cannabis in order to reach a temperature sufficient to kill spores and microorganisms. This may in turn allow the cannabis to be sterilised more quickly, and to become less damp during the steaming process thanks to the reduced time in contact with steam. This may be a particular benefit when steaming cannabis flowers and buds, which may degrade in the presence of excessive moisture.

Preferably, the steam source is adapted to provide high pressure steam to the manifold. For example, the steam source may be configured to supply steam to the manifold at a pressure of at least 1.5 bar, or at least 1.8 bar, or at least 2 bar.

The steam source may be adapted to provide superheated steam to the manifold, that is, steam heated to a temperature greater than 100 degrees centigrade. By introducing superheated steam through the lances to the cannabis plant material, the temperature of the plant material may advantageously be raised to the target temperature very quickly. This may allow the controller to reduce the steaming time required to reach the target temperature, and to sterilise the plant material, reducing the contact time between the cannabis and the steam, and reducing the potential for excessive moisture transfer to the cannabis.

The valve or valve means may also allow improved control of the steaming process by the controller, as the controller can stop and start the steam flow as desired, for example to maintain the cannabis at the target temperature or within a target temperature range.

The controller may comprise a temperature control feedback loop. The controller may be configured to control the steam supply so that the temperature of the batch of cannabis is maintained at a target temperature for a predetermined period of time, for example a time determined to be long enough to sterilise all of the cannabis material.

The controller may comprise a timer for monitoring the time during which the cannabis has been maintained at the target temperature.

The use of a temperature feedback loop may advantageously allow the controller to receive temperature signals from the temperature sensor, and to adjust the steam supply dependent on the sensed temperature of the cannabis. This may allow improved temperature control of the cannabis during steaming, which may be particularly beneficial where the cannabis is prone to degrade when subjected to high temperatures and high humidities. This also means that the controller can advantageously deliver only the minimum amount of steam needed to achieve the target temperature, to avoid excess steam raising the moisture content of the cannabis unnecessarily.

The controller may be configured to stop the supply of steam when the sensed temperature exceeds a predetermined level, or when the temperature of the cannabis has been maintained at the target temperature for a predetermined period of time.

The controller may thus operate in a temperature control mode where the temperature of the cannabis material is limited to a predetermined maximum temperature.

The controller may be programmable by a user. For example, the user may program the controller to operate in one of a variety of modes for a particular steaming cycle.

The controller may be controllable to set a target temperature, and/or a target humidity, and/or a target steaming time, and/or a target steaming pressure.

The controller may be built into the apparatus or communicate with the apparatus, for example, via a wired or wireless interface. In certain embodiments, the controller may be a mobile phone, a tablet computer, or any other handheld control device. For example, an application may be downloaded onto a mobile device allowing the device to be used as a controller. The controller can be configured to provide a user interface that allows users to input desired configurations (e.g., a target temperature, target humidity, target steaming time, and/or a target steaming pressure).

In more general terms, the controller may comprise a data processing system and/or a computer program product, for example as described in more detail further below.

The apparatus may comprise a container configured to contain the plurality of lances and, in use, the batch of cannabis.

The container is preferably thermally insulated. This advantageously improves the thermal efficiency of the apparatus by reducing heat loss from the container.

By providing at least the lances in a container which can receive a batch of cannabis for steaming, the container may confine the steam during steaming, enabling the cannabis to reach a higher temperature and/or humidity more quickly than would be possible outside a container.

The container may be sealable in a gas-tight configuration, so that steam pressure inside the container can be increased above atmospheric pressure.

This may advantageously allow the temperature of the cannabis to be increased rapidly to a level sufficient to kill mould spores and other pathogens. Cannabis may therefore be sterilized with less time in contact with moist steam, which may lead to less condensation and less moisture absorbed into the cannabis during steaming. This may be beneficial when steam cleaning certain parts of cannabis plants, like flowers or buds, where it is undesirable to raise the moisture level too high.

In a preferred embodiment, the container comprises an openable vent, the openable vent being controllable by the controller between an open state in which steam is vented from the interior of the container to the atmosphere, and a closed state in which steam cannot pass through the vent.

The controller may be programmable to open the vent as soon as steaming is complete. For example, the controller may open the vent as soon as the cannabis have reached the target temperature, or to open the vent after the cannabis have been held at the target temperature for a predetermined period of time. Opening the vent may advantageously allow steam to escape from the container, reducing the amount of condensation that accumulates on the container walls and in the cannabis material.

The vent may be continuously adjustable between its closed and open states to allow the controller to control the pressure of the steam in the container.

The apparatus may comprise a pressure sensor configured to sense a pressure inside the container, and the controller may be configured to receive signals from the pressure sensor.

The controller may be controllable to set a target steaming pressure. The controller preferably comprises a pressure control feedback loop, and is configured to control the steam supply so that the pressure of steam in the container is increased to the target steaming pressure.

The controller may be programmed to stop the supply of steam, or to open the vent, when the pressure in the container exceeds a predetermined level or reaches the target pressure.

The apparatus may comprise a humidity sensor configured to sense a humidity inside the container, and the controller may be configured to receive signals from the humidity sensor.

The humidity sensor may be provided on a probe insertable into the batch of cannabis during steaming, so that the humidity sensor might measure moisture content inside the batch during steaming, or the humidity sensor may be provided on a wall of the container containing the batch of cannabis.

The controller may be controllable to set a target humidity. The controller may comprise a humidity control feedback loop, and be configured to control the steam supply so that the humidity in the container is increased to the target humidity.

The controller may be programmed to stop the supply of steam when the sensed humidity exceeds a predetermined level or reaches the target humidity.

The controller may be configured to determine that steaming is complete based on signals from the humidity sensor. For example, the controller may open the vent as soon as the cannabis has reached the target humidity, or open the vent after the cannabis has been held at the target humidity for a predetermined period of time.

Preferably, the controller is configured to control the supply of steam and the opening, or partial opening, of the vent in response to signals from the temperature and humidity sensors, to control the temperature and humidity inside the container.

By sensing the temperature and the pressure and/or humidity in the container during steaming, the controller may advantageously control the steam supply and optionally the vent to control the steaming conditions of the cannabis. Steaming temperatures, pressures and humidities may thus be controlled to a greater extent than has been possible with prior art steamer devices intended for steaming moisture-tolerant materials such as animal fodder. This may allow the apparatus to provide different steaming conditions for different batches of cannabis, depending on the part of the plant being steamed, and to precisely control the steaming conditions.

In combination with the valve or valve means and/or the vent, the controller may ensure that the target temperature, and/or a target humidity, and/or a target steaming time, and/or a target steaming pressure is maintained for a predetermined period of time.

Typically, the parts of the Cannabis sativa plant that are intended for human consumption (for example the flowers or buds) are less moisture-tolerant and may degrade in damp conditions, the controller may be programmed to deliver high pressure steam to the container with the vent closed. This may increase the temperature of the cannabis rapidly to a temperature sufficient to kill microorganisms. During steaming, the vent and the steam supply may be controlled to maintain the target temperature without supplying excess steam which would unnecessarily increase the temperature and humidity of the cannabis. After a predetermined steaming period, the vent may then be opened to immediately vent the residual steam from the container, to reduce the quantity of condensation that is left in the cannabis. Such steaming conditions may be particularly preferable for sterilising cannabis flowers or buds, for example.

The apparatus may comprise a steam generator configured to deliver steam to the manifold, such that the steam generator forms the steam source. For example, a steam generator may be provided inside the container, or integrated with the container body or housing. In preferred embodiments, the steam generator may be housed in the base of the container, or in a wall of the container.

In a preferred embodiment, the steam generator and the manifold are provided in a shared housing. The outlet of the steam generator may form the inlet of the manifold.

By providing the steam generator and the manifold in the same housing, the distance that steam must travel between the steam generator and the manifold may be reduced or minimised. This may advantageously allow steam to be delivered to the lances at a higher pressure and/or temperature than is possible with a “remote” steam generator, as the steam does not have time to cool down, lose pressure and condense as it travels through piping from the steam generator.

In a preferred embodiment the steam generator, the manifold and the lances are all integrated into the base of a container, the container being adapted to receive a batch of cannabis on top of the manifold during steaming.

The steam generator is preferably configured to deliver steam to the lances at a pressure greater than atmospheric pressure, for example at least 1.2 bar, or at least 1.4 bar, or at least 1.6 bar, or at least 2 bar, or at least 2.5 bar.

By delivering steam at a raised pressure, the cannabis may be heatable more quickly, with less time for moisture to condense in the cannabis during steaming. This is particularly important for steaming cannabis, where it is desirable to raise the moisture level of the cannabis at little as possible, while still raising the cannabis to a sufficiently high temperature to sterilize the cannabis.

The steam generator may comprise a steam generator pressure sensor for sensing the pressure of steam in the steam generator. The controller is preferably configured to receive a signal from the steam generator pressure sensor, so that the controller can control the supply of steam to deliver steam to the cannabis at a desired pressure. For example, the controller may open the valve means to supply steam to the lances only when the steam generator pressure reaches a desired pressure of at least 1.2 bar, or at least 1.4 bar, or at least 1.6 bar, or at least 2 bar, or at least 2.5 bar.

The controller may be programmed to operate in a steam cleaning mode, or sterilisation mode, for killing mould and bacteria on cannabis, in which the controller delivers steam to raise the temperature of a batch of cannabis to a target temperature range of between 90° C. and 104° C.

In steam cleaning mode, the controller may preferably be configured to deliver steam to raise the temperature of the batch of cannabis to a temperature of at least 90° C. for a period of at least 10 minutes, or at least 15 minutes, or at least 20 minutes. Lower temperatures and steaming durations may be insufficient to ensure harmful microorganisms have been killed by steaming.

The controller may be configured to deliver steam to raise the temperature of the batch of cannabis to a maximum temperature of 104° C. for a maximum of 30 minutes. Higher temperatures and time durations may cause deterioration of the cannabis during steaming.

Preferably, the controller may deliver steam to raise the temperature of the batch of cannabis to between 90° C. and 104° C. for between 10 and 30 minutes, or between 12 and 25 minutes, or between 15 and 20 minutes. Particularly preferably, the controller may deliver steam to raise the temperature of the batch of cannabis to between 100° C. and 104° C. for between 10 and 20 minutes.

A batch of cannabis may, for example, include cannabis in baled form, for example cannabis compressed into bales or bricks, or cannabis in uncompressed or loose form, optionally held in nets, mesh bags or other containers or receptacles.

In a preferred embodiment, a bale of compressed cannabis may be steamed in the present apparatus. In another embodiment, the steamer container may be filled with a batch of loose cannabis flowers or buds. In a further embodiment, a mesh container may be filled with cannabis flowers or buds and lowered onto the steaming lances.

In preferred embodiments, the cannabis may be one or more type selected from a group of: cannabis flowers, cannabis buds, cannabis leaves, cannabis seeds, or cannabis stems.

The present invention may be particularly suitable for steam treating the parts of the Cannabis sativa plant that are intended for human consumption for medicinal or recreational purposes, either directly or after processing into cannabis-derived products. These parts of the plant, which typically contain the highest proportion of cannabinoid (for example the flowers or buds) also tend to be less moisture-tolerant and may degrade in damp conditions.

Ideally, the steam manifold and the lances, in use, are arranged to impale a bale of cannabis, by placing the bale on the lances, thus the weight of the bale assists in the process of ensuring the lances penetrate into the bulk of the bale.

Where cannabis is provided in uncompressed form in a net or bag, the lances may penetrate into the batch through holes in the net, or the bag may deform around the lances so that steam is delivered into the bulk of the batch.

Where a batch of cannabis is provided in a container, the container may be formed from mesh for example, or the container may comprise apertures adapted to receive the lances so that the container may be placed on the lances and the lances may penetrate into the batch of cannabis material held in the container.

The manifold may be adapted to rest on the ground or floor of an area, or in the base of a container, and a bale may be placed on it or the manifold may be driven into a bale. Whichever technique is used there may be a single action involved in steam treating the cannabis and once treated, any baler twine or other binding is cut and the cannabis can be distributed. Therefore, the process is quick to implement and a user is free to do other things whilst a batch of cannabis is steam treated.

The apparatus is preferably formed from a strong and heat resistant material, such as stainless steel, other metals or synthetic plastics material, which is able to withstand temperatures in excess of 110 degrees Centigrade.

The manifold ideally includes a plurality of lances, which may be in the form of prongs or spikes, each being adapted to impale a batch of cannabis so that they penetrate deep into the batch, thereby enabling steam to pass into the bulk of the batch and percolate from the centre outwards. This ensures deep and thorough heating—and thus steam treatment—of the cannabis.

There may be two, but ideally there are three, four, five or more lances arranged in an array so that the lances are in a form that enhances even distribution of steam throughout the batch of cannabis. Again ensuring deep and thorough heating—and thus steam treatment—of the cannabis.

The lances may all be the same length, or height.

Alternatively, the plurality of lances may have different lengths. Lances of different lengths arranged on the same manifold may advantageously provide improved steam distribution throughout a batch of cannabis by distributing steam to different areas within the batch. This may be particularly beneficial where the cannabis is provided in a bale, so that steam must percolate throughout a large volume of cannabis material.

The apparatus may comprise a lance with a first length, a lance having a second length greater than the first length, and a lance having a third length greater than the second length.

In a preferred embodiment, the apparatus may comprise at least two lances with a first length, and at least one lance having a second length greater than the first length. The apparatus may further comprise at least one lance having a third length greater than the second length. These lances may allow steam to be distributed at different heights within a batch of cannabis, for example a bale of cannabis, so that steam is distributed throughout the batch.

There may be two, but ideally there are three, four, five or more lances arranged in an array so that the lances are in a form that enhances even distribution of steam throughout the batch. Again ensuring deep and thorough heating—and thus steam treatment—of the cannabis. In an example, the array may comprise at least two lances of the first length, at least one lance of the second length and at least one lance of the third length, the lengths as described above.

The size of the manifold, and the number of lances in the array, may be chosen to correspond to the capacity of the container, and therefore the volume of cannabis the apparatus is configured to steam. Alternatively, the apparatus may comprise a plurality of manifolds. Each manifold may comprise a plurality of lances arranged in an array, as described above.

Ideally, the apparatus includes a heater which has an immersion element and is adapted for use with either 240 Volts or 110 Volts. The heater generates steam in the well-known manner.

In a second aspect, the invention may provide a method of steaming cannabis, comprising the steps of:

inserting a plurality of lances into a batch of cannabis;

delivering steam from a steam source, through a manifold, and out of the lances into the batch of cannabis;

sensing a temperature of the batch of cannabis; and

controlling the supply of steam to the lances in response to the sensed temperature, to raise the temperature of the cannabis to a target temperature.

The method of steaming cannabis may comprise the use of the apparatus described above as the first aspect of the invention. Thus, the step of controlling the supply of steam may be carried out by a controller.

In a preferred embodiment of the method, steam may be delivered to the cannabis at a pressure greater than atmospheric pressure, for example at least 1.2 bar, or at least 1.4 bar, or at least 1.6 bar, or at least 2 bar, or at least 2.5 bar.

The steam supply may be controlled so that the temperature of the batch of cannabis is maintained at a target temperature for a predetermined period of time.

The supply of steam may be stopped when the sensed temperature exceeds a predetermined level.

In a preferred embodiment, the plurality of lances and the batch of cannabis are placed in a container before steam is delivered from the steam source. The container may be sealed in a gas-tight configuration while the steam is delivered to the cannabis, so that steam pressure inside the container increases above atmospheric pressure.

The method may comprise the step of sensing a pressure inside the container, and/or sensing a humidity inside the container.

The method may comprise the step of controlling the supply of steam to reach a predetermined steam pressure inside the container.

The method may comprise the step of opening a vent in the container to vent steam from the interior of the container to the outside atmosphere when the pressure, or the temperature, or the humidity in the container exceeds a predetermined level.

In a further aspect, the invention may provide a method of steam cleaning cannabis, comprising the method of the second aspect of the invention, in which the supply of steam is controlled to raise the temperature of a batch of cannabis to a target temperature range of between 90° C. and 104° C.

In order to steam clean the cannabis the supply of steam may be controlled to raise the temperature of the batch of cannabis to a temperature of at least 90° C. for a period of at least 10 minutes, or at least 15 minutes, or at least 20 minutes.

The supply of steam may be controlled to raise the temperature of the batch of cannabis to a maximum temperature of 104° C. for a maximum of 30 minutes.

The features described above in relation to one aspect of the invention are applicable to any of the other aspects of the invention.

Preferred embodiments of the invention will now be described, by way of examples only, and with reference to the Figures in which:

### BRIEF DESCRIPTION OF THE FIGURES

FIG. 1 is an overall diagrammatical view of one embodiment of the invention and illustrates the principle of operation;

FIG. 2A is an overall view of an example of a manifold and lances;

FIG. 2B is an overall view of an alternative embodiment of manifold and lances having different lengths;

FIG. 3 is a partially transparent illustration of a first embodiment of the invention in use;

FIG. 4 is a partially transparent illustration of a second embodiment of the invention in use; and

FIG. 5 is a schematic diagram of a further embodiment of the invention.

### DETAILED DESCRIPTION OF PREFERRED EMBODIMENTS OF THE INVENTION

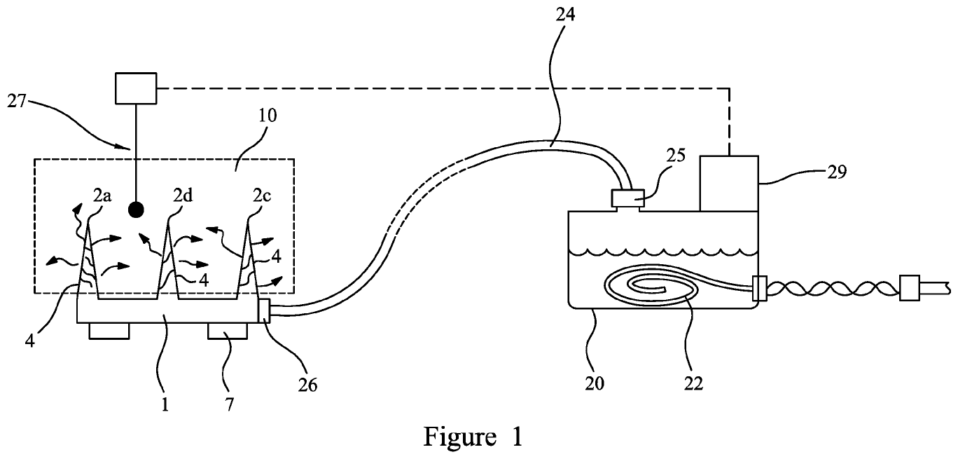

Referring to FIG. 1 there is shown in general an embodiment of an apparatus for steam treatment of cannabis comprising a water reservoir 20 in which is located a heating element 22. The water reservoir, or steam generator, can also be a sealed vessel and thus capable of heating water to more than its normal boiling point. A high pressure flexible hose 24, which is ideally insulated, conducts steam from the reservoir to a manifold, ideally via a flexible or universal joints 25 and 26. The manifold 1 distributes the steam into a batch of harvested cannabis 10.

A temperature sensor probe 27 is provided to measure a temperature inside the batch of cannabis during steaming, and to communicate temperature signals to a controller 29.

The reservoir or steam generator optionally includes conventional safety equipment such as thermostatic settings, boil dry warning and residual current detectors (RCD) for use in damp and outdoor environments.

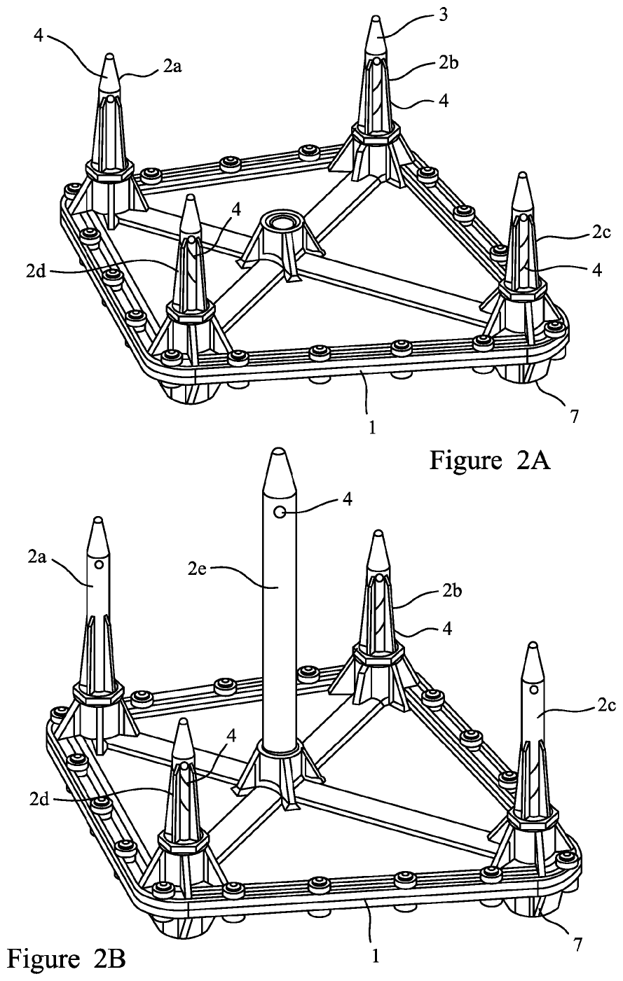

FIG. 2 shows a manifold, which is in the form of a generally square frame and has passages formed therein for distribution of steam. The manifold 1 is fitted with a number of substantially vertical lances 2, which are in communication with the passages. Each lance 2 has a pointed end 3 for ease of penetration into a batch of cannabis 10. Lances 2 have apertures 4, extending a proportion of their length, for the release and distribution of steam and condensed steam into the centre of the batch of cannabis.

The supply of steam may be switched on or off by the controller 29. The controller is preferably programmable by a user, and is configured to control steam supply to the cannabis, and therefore to control the steaming temperature and duration of the steaming.

The controller may be built into the apparatus or communicate with the apparatus, for example, via a wired or wireless interface. In certain embodiments, the controller may be a mobile phone, a tablet computer, or any other handheld control device. For example, an application may be downloaded onto a mobile device allowing the device to be used as a controller. The controller can be configured to provide a user interface that allows users to input desired configurations (e.g., a target temperature, target humidity, target steaming time, and/or a target steaming pressure).

The controller may comprise a data processing system, which may include one or more processors (e.g., a general purpose microprocessor and/or one or more other data processing circuits, such as an application specific integrated circuit (ASIC), field-programmable gate arrays (FPGA's), and the like); a network interface for connecting the controller to a network; and a local storage unit, which may include one or more non-volatile storage devices and/or one or more volatile storage devices (e.g., random access memory (RAM)). In embodiments where the controller includes a general purpose microprocessor, a computer program product may be provided. The computer program product may include a computer readable medium (CRM) storing a computer program comprising computer readable instructions. The CRM may be a non-transitory computer readable medium, such as, but not limited to magnetic media (e.g., a hard disk), optical media (e.g., a DVD), memory devices (e.g., random access memory), and the like. In some embodiments, the computer readable instructions are configured such that when executed, they cause the controller to perform tasks described herein. In other embodiments, the controller may be configured to perform tasks described herein without the need for code. For example, the data processing system may consist merely of one or more ASICs.

The apertures 4 may be vertically disposed or they may be in the form of slits or slots, extending lengthwise or helically about circular lances 2. Alternatively, the apertures may take the form of holes formed in the lances so that the steam and condensed steam permeate outwards to the extremities of the batch of cannabis ensuring full effectiveness and contact of the steam throughout the batch. It has been found that with use of the manifold the temperature of a bale of cannabis may exceed 100 degrees Centigrade.

As a result of the lances 2a, 2b, 2c and 2d, steam is introduced into the centre of the bale or receptacle of the cannabis material by placing the batch of cannabis onto the manifold 1. Alternatively, the manifold 1 can be forced into a batch of cannabis 10 from the side or above. In whichever orientation the lances penetrate so as ensure steam reaches all of the batch. If the manifold is arranged to rest on the ground during operation, an optional foot or feet 7 may be provided or formed on the manifold to prevent damage to it, for example by shock loading that may occur when a bale of compacted cannabis is dropped onto the lances.

In an alternative embodiment one or more steam distribution manifolds 1 of various lengths (to accommodate different size bales and receptacles to ensure effective central penetration) is provided. Lances 2 may be of different lengths and optionally these may be removable and fitted onto a common manifold. Thus for example in the event of damage to a lance, it may be removed and replaced with a similar lance. Alternatively, where the manifold is required for use with larger bales, longer lances may be fitted to the manifold.

FIG. 2B shows an embodiment in which five lances 2a, 2b, 2c, 2d and 2e are connected to a manifold 1. In order to provide good distribution of steam at different heights in a bale of plant fibres, the lances have different lengths, so that they extend to different heights above the manifold 1. As shown in FIG. 2B, two lances 2b and 2d on opposite sides of the manifold have a first length or height, two lances 2a and 2c on opposite sides of the manifold have a second length or height, which is greater than the first length, and a lance 2e in the centre of the manifold has a third length or height which is greater than the lengths of the other lances. Apart from the lance lengths, the design of each lance is substantially the same as described above in relation to FIG. 2A. When a large net bag or bale of cannabis is placed on the manifold 1 for steaming, the lances will penetrate the net bag or bale so that the tips of the lances, and therefore the steam apertures, extend to different heights inside the batch of cannabis. This means that steam is delivered to different points inside the batch, providing more uniform steaming throughout the batch than would be possible with lances of uniform length.

Steam and condensed steam permeate through the batch of cannabis increasing the temperature of the cannabis to between 90 and 105 degrees Centigrade (depending upon ambient temperature) killing thermophilic and mesophilic mould spores and other living organisms as mentioned above and effectively steam treating the fodder as well as dampening dust spores thus restricting their ability to become airborne.

The steam is distributed from the reservoir, via the hose and through the lances and where the steam condenses the water content is absorbed, in the majority, by the cannabis leaving it damp. As the moisture content within the cannabis increases, the temperature rises exponentially due to the increased efficiency of water as a heat conducting medium within the plant material, compared to air in the cannabis' dry state.

For certain parts of the cannabis plant, however, for example cannabis flowers and buds, it is undesirable to introduce excessive moisture through condensation.

In prior art fodder steaming devices, the lack of control system means that fodder was typically steamed continuously for a long period, during which the fodder absorbed moisture and the temperature of the fodder increased exponentially. The temperature probe 27 and controller of the present device, however, allow more precise control so that the cannabis can be heated to and maintained at a desired target temperature for the desired duration. The apparatus may therefore achieve the temperature necessary to kill pathogens without continuing to add more steam which would undesirably increase the temperature and moisture content of the cannabis further than needed.

Depending on the programming of the controller, the cannabis may be exposed to continuous steam from the apparatus, or the steam supply may be intermittently stopped and started to maintain the cannabis at a desired temperature, for example. In order to kill microorganisms the cannabis material may typically be heated to a temperature of at least 90 degrees centigrade for not less than 10 minutes.

The aforementioned apparatus can be used either in open space or within an enclosed environment, such as horse box, stable or barn.

In a preferred embodiment the manifold 1 and lances 2 are provided inside a designated sealable container 30, which is preferably insulated and gas-tight so that steam pressure may be built up within the container.

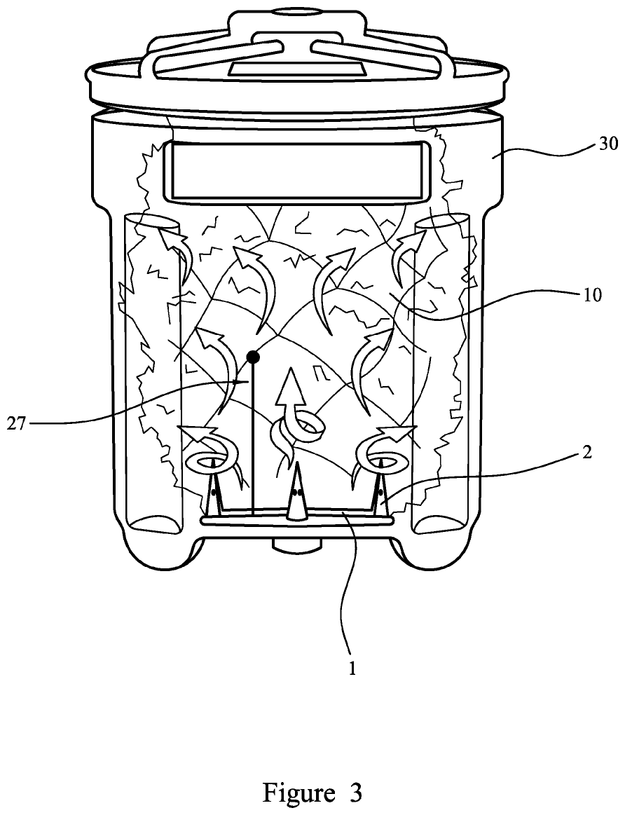

FIG. 3 illustrates a first embodiment of a steaming apparatus in which a batch of uncompressed cannabis contained in a net 10 are steamed in a container 30. A manifold 1 is positioned in the base of the container 30, so that in use the batch of cannabis is lowered down onto the lances 2 so that they penetrate into the batch of cannabis. A temperature probe 27 also projects upwards from the base of the container so that it penetrates into the batch of cannabis to measure the temperature inside the batch during steaming. A humidity sensor (not shown) is provided in a container wall, which communicates humidity signals to the controller.

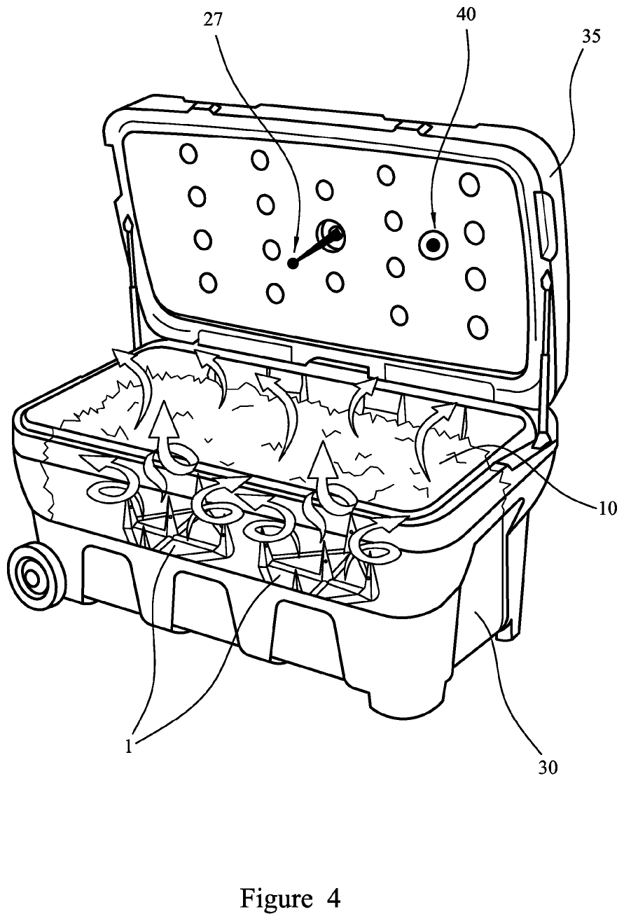

FIG. 4 shows an alternative embodiment of the steaming apparatus including a larger container 30, which contains two manifolds 1 in its base. In this embodiment, the temperature probe 27 is provided on the lid 35 of the container. When closed, the lid 35 preferably forms a gas-tight seal with the container walls so that the steam pressure in the container can be increased to a desired pressure. A pressure sensor (not shown) is provided in a container wall, which communicates pressure signals to the controller. A humidity sensor is also provided in the container.

A closable vent 40 is provided in the container lid 35 for releasing steam from the interior of the container. Opening and closing of the vent is controllable by the controller, either in response to a threshold pressure, temperature or humidity being reached, or to release steam from the container at the end of the desired steaming time.

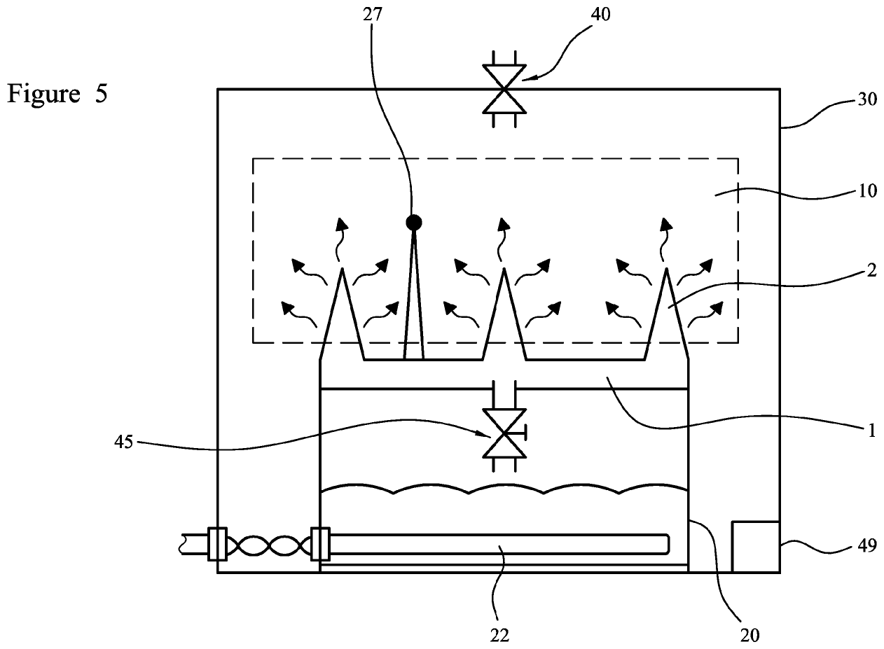

In the embodiment of the apparatus shown in FIG. 5, the manifold 1 is positioned directly above the boiler 20, with a valve 45 controlling the flow of steam from the boiler to the manifold. The opening and closing of the valve 45 is controllable by a controller 49. As the boiler and the manifold are adjacent to one another, high pressure steam may be generated in the boiler and supplied to the manifold without having the chance to cool down and condense in a connecting hose 24. This may be particularly preferred for supplying high pressure steam to the batch of cannabis 10 in the interior of the container 30, as it may allow rapid heating of the cannabis to kill spores and microorganisms. Once the cannabis has been heated to sufficient temperature for long enough to kill pathogens, the controller preferably opens the vent 40 to release the residual steam in the container and reduce the amount of condensation left in the cannabis.

1. An apparatus for steam treating cannabis, the apparatus comprising:
a steam manifold that is adapted to receive steam from a steam source and to distribute steam to a plurality of lances, the lances being configured to deliver steam into the interior volume of a batch of cannabis;
a temperature sensor for sensing a temperature of the batch of cannabis; and
a controller configured to receive a signal from the temperature sensor, and to control the supply of steam to the lances to raise the temperature of the cannabis to a target temperature.

2. An apparatus according to claim 1, in which the apparatus comprises valve means controllable to prevent or allow the passage of steam from the steam source to the lances, and in which the valve means are controllable by the controller.

3. An apparatus according to claim 1 or 2, in which the controller comprises a temperature control feedback loop, and is configured to control the steam supply so that the temperature of the batch of cannabis is maintained at a target temperature for a predetermined period of time.

4. An apparatus according to claim 1, 2 or 3, in which the controller is configured to stop the supply of steam when the sensed temperature exceeds a predetermined level.

5. An apparatus according to any preceding claim, in which the apparatus comprises a container configured to contain the plurality of lances and, in use, the batch of cannabis.

6. An apparatus according to claim 5, in which the container is sealable in a gas-tight configuration, so that steam pressure inside the container can be increased above atmospheric pressure.

7. An apparatus according to claim 5 or 6, in which the container comprises an openable vent, the openable vent being controllable by the controller between an open state in which steam is vented from the interior of the container to the atmosphere, and a closed state in which steam cannot pass through the vent.

8. An apparatus according to claim 5, 6 or 7, in which the apparatus comprises a pressure sensor configured to sense a pressure inside the container, and in which the controller is configured to receive signals from the pressure sensor.

9. An apparatus according to claim 8, in which the controller comprises a pressure control feedback loop, and is configured to control the steam supply so that the pressure of steam in the container is increased to a target level.

10. An apparatus according to claim 8 or 9, in which the controller is programmed to stop the supply of steam, or to open the vent, when the pressure in the container exceeds a predetermined level.

11. An apparatus according to any preceding claim, in which the apparatus comprises a humidity sensor configured to sense a humidity inside the container, and in which the controller is configured to receive signals from the humidity sensor.

12. An apparatus according to claim 11, in which the controller comprises a humidity control feedback loop, and is configured to control the steam supply so that the humidity in the container is increased to a target level.

13. An apparatus according to claim 11 or 12, in which the controller is programmed to stop the supply of steam when the sensed humidity exceeds a predetermined level.

14. An apparatus according to any preceding claim, in which the apparatus comprises a steam generator configured to deliver steam to the manifold.

15. An apparatus according to claim 14, in which the steam generator and the manifold are provided in a shared housing.

16. An apparatus according to claim 14 or 15, in which the steam generator is configured to deliver steam to the lances at a pressure greater than atmospheric pressure, for example at least 1.2 bar, or at least 1.4 bar, or at least 1.6 bar, or at least 2 bar.

17. An apparatus according to any preceding claim, in which the controller is programmed to operate in a steam cleaning mode for killing bacteria on cannabis, in which controller delivers steam to raise the temperature of a batch of cannabis to a target temperature range of between 90° C. and 104° C.

18. An apparatus according to claim 17, in which the controller is configured to deliver steam to raise the temperature of the batch of cannabis to a temperature of at least 90° C. for a period of at least 10 minutes, or at least 15 minutes, or at least 20 minutes.

19. An apparatus according to claim 17 or 18, in which the controller is configured to deliver steam to raise the temperature of the batch of cannabis to a maximum temperature of 104° C. for a maximum of 30 minutes.

20. A method of steaming cannabis, comprising the steps of:
inserting a plurality of lances into a batch of cannabis;
delivering steam from a steam source, through a manifold, and out of the lances into the batch of cannabis;
sensing a temperature of the batch of cannabis; and
controlling the supply of steam to the lances in response to the sensed temperature, to raise the temperature of the cannabis to a target temperature.

21. A method according to claim 20, in which the steam is delivered to the cannabis at a pressure greater than atmospheric pressure, for example at least 1.2 bar, or at least 1.4 bar, or at least 1.6 bar, or at least 2 bar.

22. A method according to claim 20 or 21, in which the cannabis is provided as a bale of cannabis, or as a batch in a net, bag, or container.

23. A method according to claim 20, 21 or 22, in which the steam supply is controlled so that the temperature of the batch of cannabis is maintained at a target temperature for a predetermined period of time.

24. A method according to any of claims 20 to 23, in which the supply of steam is stopped when the sensed temperature exceeds a predetermined level.

25. A method according to any of claims 20 to 24, in which the plurality of lances and the batch of cannabis are placed in a container.

26. A method according to claim 25, in which the container is sealed in a gas-tight configuration while the steam is delivered to the cannabis, so that steam pressure inside the container increases above atmospheric pressure.

27. A method according to claim 25 or 26, in which the method comprises the step of sensing a pressure inside the container, and/or sensing a humidity inside the container.

28. A method according to claim 25, 26 or 27, comprising the step of controlling the supply of steam to reach a predetermined steam pressure inside the container.

29. A method according to any of claims 25 to 28, comprising the step of opening a valve in the container to vent steam from the interior of the container to the outside atmosphere when the pressure, or the temperature, or the humidity in the container exceeds a predetermined level.

30. A method of steam cleaning cannabis, comprising the method of any of claims 20 to 29,
in which the supply of steam is controlled to raise the temperature of a batch of cannabis to a target temperature range of between 90° C. and 104° C.

31. A method according to claim 30, in which the supply of steam is controlled to raise the temperature of the batch of cannabis to a temperature of at least 90° C. for a period of at least 10 minutes, or at least 15 minutes, or at least 20 minutes.

32. A method according to claim 30 or 31, in which the supply of steam is controlled to raise the temperature of the batch of cannabis to a maximum temperature of 104° C. for a maximum of 30 minutes.

33. An apparatus or method according to any preceding claim, in which the cannabis is cannabis for human consumption for medicinal or recreational purposes, preferably selected from the list of: cannabis flowers, cannabis buds, cannabis leaves, cannabis stems and cannabis seeds.
